(19) United States
(12) Patent Application Publication
(10) Pub. No.: US 2012/0143151 A1
(43) Pub. Date: Jun. 7, 2012

(54) NEEDLE SHIELD AND A WINGED NEEDLE ASSEMBLY

(75) Inventors: Boon Tong LOW, Singapore (SG); Ai Ling TEO, Singapore (SG); Heng Jew LIM, Singapore (SG)

(73) Assignee: JMS SINGAPORE PTE LTD, Singapore (SG)

(21) Appl. No.: 12/959,555

(22) Filed: Dec. 3, 2010

(51) Int. Cl. A61M 5/00; B29C 67/00

(52) U.S. Cl. 604/263; 264/299

(57) ABSTRACT

A needle shield having a front end opening through which a winged needle as a whole is retracted and a base end opening through which a flexible tube connected to the winged needle is passable. The needle shield includes a winged needle retention means projecting into the interior side of the needle shield to define one or more traps, the winged needle retention means receiving, into the one or more traps, portions of one or more wings of the winged needle for preventing protrusion of the winged needle past the winged needle retention means when the winged needle is translated within said needle shield towards the front end opening of the needle shield from a retracted state of the winged needle.

### FIELD OF INVENTION

The present invention broadly relates to a needle shield and a winged needle assembly for preventing inadvertent needle punctures, and, more particularly, to a needle shield for preventing protrusion of a winged needle from the needle shield after the needle is removed from a needle user. The present invention also relates broadly to methods of manufacturing the needle shield and the winged needle assembly.

### BACKGROUND

Needles are used in a variety of settings, including medical. Such medical uses of needles include haemodialysis and blood collection. Unfortunately, inadvertent needle mispiercings can be dangerous to medical professionals and others handling used and exposed needles. Infection due to inadvertent piercings with used needles have become a serious issue. Needles may spread disease to those that are inadvertently exposed to the contents of needles when accidentally punctured with infected needles. Proper handling of needles will reduce the possibility of infecting persons that come in contact with used needles. Thus, means for preventing inadvertent piercings have attracted much attention of medical professionals.

Simple, conventional needle covers that operate as a cap over the needles have not been adopted in some medical facilities because of inconvenience, the increase in operators' work time, the likelihood of mispiercings while attempting to place the cap on the needle, and the costs of the extra caps. Winged needles with needle shields have been introduced as an alternative that is less likely to cause inadvertent puncture during the retention of the needle after usage. As the injection needle or indwelling needle, a winged needle which is formed with a wing body assembled to a needle is widely used to facilitate gasping and indwelling. Retaining winged needles within needle shields would prevent inadvertent needle mispiercings. To retain winged needles within needle shields, several solutions have been proposed.

One approach is the use of slidable slots located along the side walls of the needle shield. The wings of a winged needle extend outside the needle shield through the slidable slots. After using the needle, the user slides the needle back towards the back end of the needle shield, putting the winged needle in a retracted state. Once the needle has been pulled towards the back end of the needle shield, there is a cut-out tab with two shoulders in the slidable slot that prevents the wings from moving back or forward, thus preventing re-protrusion. Unfortunately, the wings can pop out from between the shoulders back onto the slidable slot, allowing the winged needle to move towards the front end opening, re-exposing the used needle.

Another proposed approach is to shape the side faces of the needle shield so that the needle shield has a convex shape. As the winged needle is retracted into the needle shield, the wings of the winged needle are folded and tucked in when the wings are pulled against the front end opening of the needle shield, and the folded wings slip into the convex side faces of the needle shield. The friction between the wings and the side faces creates some resistance that retains the wings to some degree, but the wings may still remain movable to an extent that may cause re-protrusion.

### SUMMARY

One aspect of the present invention provides a needle shield having a front end opening through which a winged needle as a whole is retracted and a base end opening through which a flexible tube connected to said winged needle is passable, comprising winged needle retention means projecting into the interior side of said needle shield to define one or more traps, said winged needle retention means receiving, into said one or more traps, portions of one or more wings of said winged needle for preventing protrusion of said winged needle past said winged needle retention means when said winged needle is translated within said needle shield towards said front end opening of said needle shield from a retracted state of said winged needle.

Said winged needle retention means of said needle shield may project from at least one of a side face, a top face, or a bottom face of said needle shield into the interior of said needle shield to define one or more traps.

Said one or more traps of said needle shield may each be formed by a tentacle joined to one of said side face, said top face, or said bottom face of said needle shield.

At least one of said one or more traps of said needle shield may be formed by creating an acute angle.

Said needle shield may include a propping means propping said needle shield away from skin of a user of said needle shield.

Said propping means of said needle shield may be a protrusion with radius edges protruding from an exterior side of a bottom face of said needle shield.

Said base end opening of said needle shield grippingly may secure a needle hub or a wing base of said winged needle when said winged needle is translated within said needle shield towards said base end opening.

A further aspect of the present invention provides a winged needle assembly comprising: a winged needle; a flexible tube connected to said winged needle, and a needle shield integrally formed with said flexible tube and having a front end opening through which said winged needle as a whole is retracted and a base end opening through which said flexible tube connected to said winged needle is passable, wherein one or more tentacles project into the interior side of said needle shield to define one or more traps, wherein said one or more tentacles are capable of receiving, into said one or more traps, portions of one or more wings of said winged needle, wherein said needle shield prevents protrusion of said winged needle past said one or more tentacles when said winged needle is translated within said needle shield towards said front end opening of said needle shield from a retracted state of said winged needle.

A further aspect of the present invention provides a method of manufacturing a needle shield having a front end opening through which a winged needle as a whole is retracted and a base end opening through which a flexible tube connected to said winged needle is passable, the method comprising the steps of:

forming needle retention means projecting into the interior side of said needle shield to define one or more traps, said winged needle retention means receiving, into said one or more traps, portions of one or more wings of said winged needle for preventing protrusion of said winged needle past said winged needle retention means when said winged needle is translated within said needle shield towards said front end opening of said needle shield from a retracted state of said winged needle.

A further aspect of the present invention provides a method of manufacturing a winged needle assembly, the method comprising the steps of:

providing a winged needle; providing a flexible tube connected to said winged needle; and manufacturing a needle shield having a front end opening through which said winged needle as a whole is retracted and a base end opening through which said flexible tube connected to said winged needle is passable,
wherein manufacturing the needle shield comprises forming needle retention means projecting into the interior side of said needle shield to define one or more traps,
said winged needle retention means receiving, into said one or more traps, portions of one or more wings of said winged needle for preventing protrusion of said winged needle past said winged needle retention means when said winged needle is translated within said needle shield towards said front end opening of said needle shield from a retracted state of said winged needle.

The method may further include wherein said needle shield is manufactured in a single solid piece form as a whole by moulding.

The method may further include wherein said needle shield is manufactured through split-mould members integrated by at least one hinge member.

### BRIEF DESCRIPTION OF THE DRAWINGS

Embodiments of the invention will be better understood and readily apparent to one of ordinary skill in the art from the following written description, by way of example only, and in conjunction with the drawings, in which:

FIG. 1 illustrates a needle shield connected with a winged needle, according to an embodiment.

FIG. 2a and FIG. 2b illustrate perspective views of the needle shield, according to an embodiment.

FIG. 3 illustrates the bottom portion of the needle shield, according to an embodiment.

FIG. 4a, FIG. 4b, and FIG. 4c illustrate a side view schematic, a top view schematic, and a front view schematic of the needle shield, respectively, according to an embodiment.

FIG. 4d illustrates a schematic top view of the needle shield 100, according to one embodiment.

FIG. 4e, FIG. 4f, FIG. 4g illustrate an overhead view and schematic diagrams to show dimensions of the needle shield, according to an embodiment.

FIG. 5a, FIG. 5b, and FIG. 5c illustrate tentacles located at the top face of the needle shield, projecting from the top face down into the interior of the needle shield, according to another embodiment.

FIG. 5d, FIG. 5e, FIG. 5f, and FIG. 5g illustrate tentacles located at the bottom face of the needle shield, projecting from the bottom face up into the interior of the needle shield, according to another embodiment.

FIG. 6 illustrates the needle shield with anti-skid notches, according to an embodiment.

FIG. 7a illustrates the needle shield securing a winged needle formed with a rotating hub, according to an embodiment.

FIG. 7b illustrates the needle shield securing a winged needle formed with a wing base, according to an embodiment.

FIG. 7c illustrates the needle shield with additional enhanced features, according to another embodiment.

FIG. 8 illustrates a flow diagram for manufacturing a winged needle with a wing-trapping type inadvertent puncture protector, according to another embodiment.

FIG. 9 illustrates a flow diagram for manufacturing a winged needle assembly, according to an example embodiment.

### DETAILED DESCRIPTION

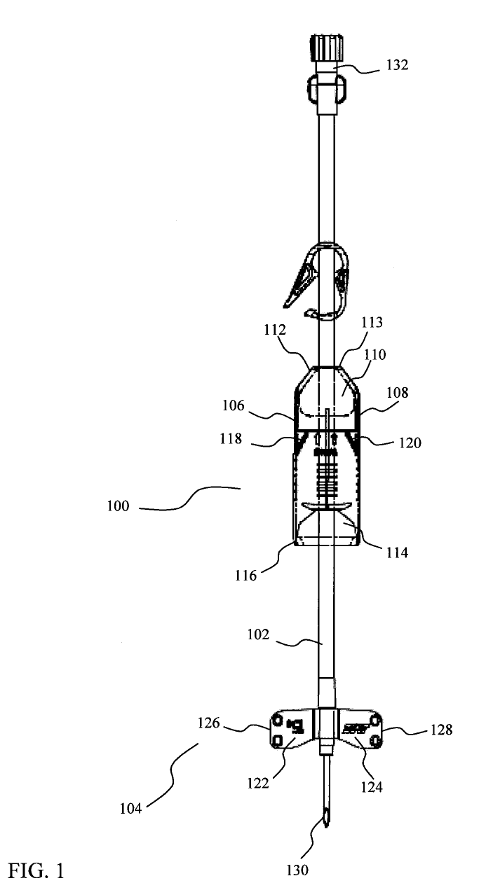

Embodiments of the invention will be discussed hereinafter with reference to the figures. FIG. 1 illustrates a needle shield with a winged needle, according to an embodiment. In FIG. 1, a needle shield 100 is loosely fitted to a flexible tube 102. The flexible tube 102 is connected to a winged needle 104. Needle shield 100 is capable of retaining the winged needle 104 as a whole. The needle shield has a configuration consisting of a substantially flat bottom face, two side faces 106, 108 connected to the bottom face, and a top face 110 connected to the two side faces. Located at base end 112 is a base end circular opening 113 through which a flexible tube connected to the winged needle is passable. In different embodiments, the base end circular opening 113 can resemble an oval shape, square shape, star shape, or any other shape. The size of the base end circular opening 113 may be approximately the same size as, or slightly smaller than, the size of the flexible tube 102. The base end circular opening 113 may be slightly smaller than the size of the flexible tube 102 so that the flexible tube 102 is slightly supported by the base end circular opening 113 while passage of the flexible tube 102 is not hindered. A front end opening 114 of needle shield 100 is located at a front end 116 of needle shield 100. Winged needle 104 may be retracted as a whole through front end opening 114.

Tentacles 118, 120 extending into the interior of needle shield 100 prevent re-protrusion of winged needle 104. Winged needle 104 comprises wings 122, 124, each wing having a wing end 126, 128, respectively. Wings 122, 124 are trapped by tentacles 118, 120 when winged needle 104 is translated towards front end 116, as will be described in further detail below. When wings 122, 124 are trapped by tentacles 118, 120, winged needle 104 cannot protrude out of needle shield 100.

A needle tube 130 protrudes from winged needle 104 for injection or extraction of substances. For example, blood extracted from a patient through needle tube 130 moves through flexible tube 102 to tail 132, which is connected to a container, such as a blood pouch. Likewise, blood and pharmaceutical liquids may move from tail 132 through flexible tube 102 to be injected into the patient via needle tube 130.

The needle tube 130 of the winged needle 104 is punctured into the body of a patient, with needle shield 100 being loosely fitted to flexible tube 102 of the winged needle 104 to which the flexible tube 102 is connected. While the winged needle 104 is being punctured into the patient at an access site, the wings 122, 124 are usually fixed to the skin of the patient with a tape or the like. Upon completion of a treatment or therapeutic procedure, the tape is removed and the winged needle 104 is withdrawn from the patient body while pressing the access site with sterilising cotton. The needle tube 130 is retained in the needle shield 100 after the withdrawal of the winged needle 104, and the withdrawal operation and retaining operation into the needle shield 100 are conducted by pulling the tube towards the base end 112 while pressing a part of the needle shield 100 with a finger. Winged needle 104 is translated towards base end 112 until winged needle 104 is in a retracted position and cannot protrude out of needle shield 100.

Once winged needle 104 is retracted into a retracted state, winged needle 104 will advantageously be prevented from protrusion out of the needle shield 100 by traps in the interior of the needle shield 100. Traps are examples of winged needle retention means. Traps are created by physical extensions, called tentacles, that project from a wall of the needle shield 100 into the interior of the needle shield 100. Once the winged needle 104 has been retracted towards the base end 112 of the needle shield 100, behind the location of the traps, the traps prevent re-protrusion of the winged needle 104. The traps may resemble slots that wings 122, 124 of the winged needle 104 naturally slide into when the winged needle 104 is translating towards a front end 116 of the needle shield 100. When the wings of the winged needle 104 slide into the slot-like traps, the winged needle 104, including the wings 122, 124 and the needle tube 130, is prevented from protruding out of the needle shield 100, thereby preventing inadvertent needle punctures.

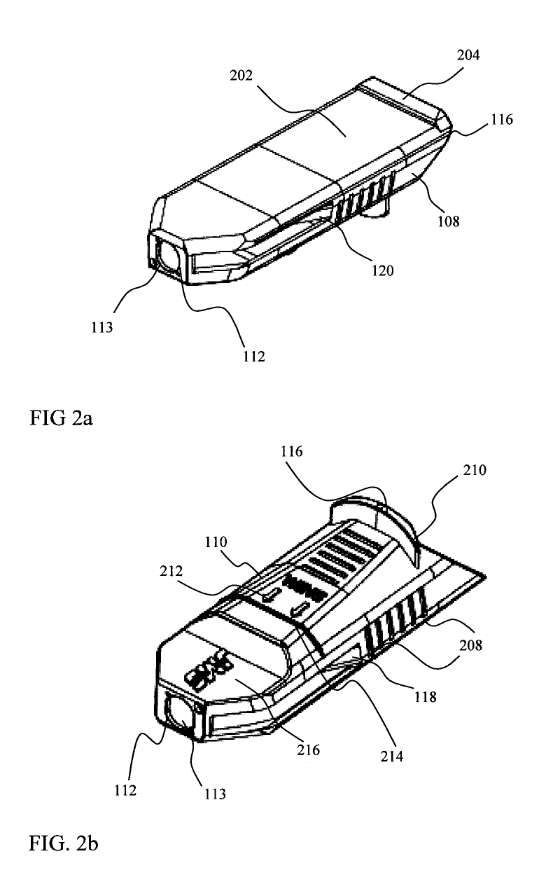

FIG. 2a and FIG. 2b illustrate perspective views of needle shield 100, according to an embodiment. FIG. 2a depicts the exterior of a bottom face 202 of needle shield 100. Base end 112 is also depicted in configuration with bottom face 202 and side face 108. Located at base end 112 is the base end circular opening 113 through which the flexible tube 102 (FIG. 1) connected to the winged needle 104 (FIG. 1) is passable. A foot 204 is connected to or formed integrally with the exterior of bottom face 202 at front end 116. Foot 204 reduces uncomfortable skin contact with the potentially sharp edges of front end 116 by propping front end 116 away from the skin of the user. For example, foot 204 may be a protrusion with radius edges protruding from the exterior side of the bottom face 202 of the needle shield 100. Needle shield 100 also comprises top face 110. Finger grips 208 and arcuate projection 210 allow the user to grip needle shield 100 tightly when utilising winged needle 104 with the patient, such as when collecting blood samples. Although reference is made to “user” and “patient”, in some circumstances, the user and the patient may be the same person. In other situations, the user may be a medical professional or some other person that is not the patient.

After using the needle, in order to withdraw winged needle 104 (FIG. 1) into a retracted state, the user may grip needle shield 100 with one hand, while pulling back on flexible tube 102 with the other hand. Arcuate projection 210 on top face 110 allows the user to pull flexible tube 102 back towards base end 112 while pressing the top face 110 of the needle shield 100. In addition, arrow markers 212 indicate the direction for translating winged needle 104 to the retracted position. In some embodiments, a safety line 214 located on top face 110 provides an indication to the user as to how far back towards base end 112 the user must translate winged needle 104, in order to secure winged needle 104 in the retracted position. The winged needle 104 may be retained into the needle shield 100 in this manner. Winged needle 104 is thus securely prevented from re-protrusion by tentacles 118, 120.

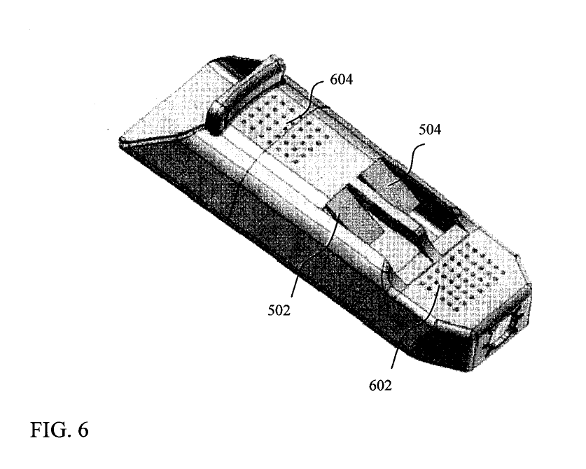

Finger grips 208 on the exterior side of side faces 106, 108 also allow the user to grip needle shield 100 tightly while winged needle 104 is withdrawn into needle shield 100. Finger grips 208 may be raised lines or grooves, as illustrated in FIG. 2a and FIG. 2b. Finger grips 208 may also be, for example, raised dots, as illustrated in FIG. 6. A recess 216 as illustrated in FIG. 2b is provided on the base end of the needle shield 100 as a holding member for user to position the thumb or finger on needle shield 100 for withdrawal of winged needle 104 into the needle shield 100.

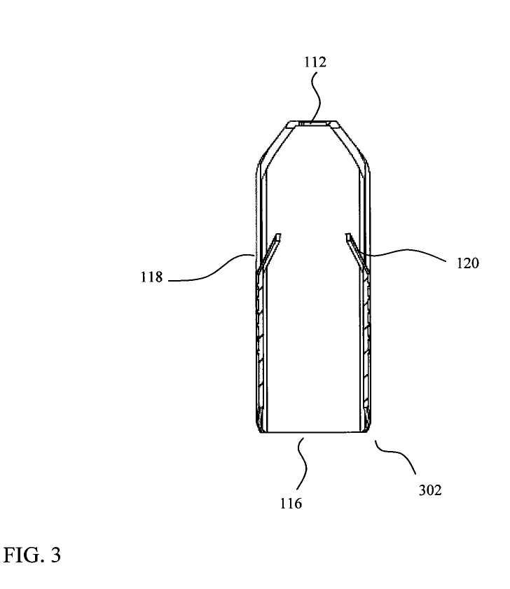

FIG. 3 illustrates the bottom portion 302 of the needle shield, according to an embodiment. Tentacles 118 and 120 are clearly depicted projecting into the interior of needle shield 100, forming traps. In the illustration of FIG. 3, for example, winged needle 104 (FIG. 1) is withdrawn from front end 116 back towards base end 112, past tentacles 118 and 120.

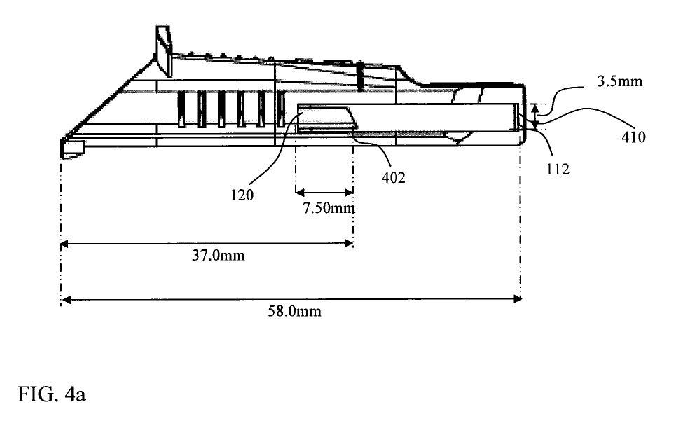

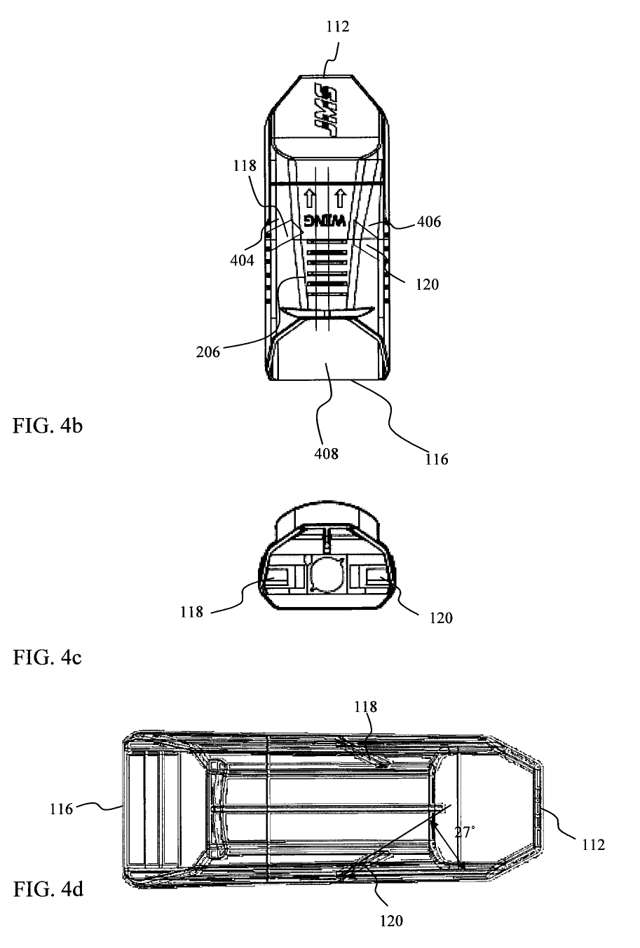

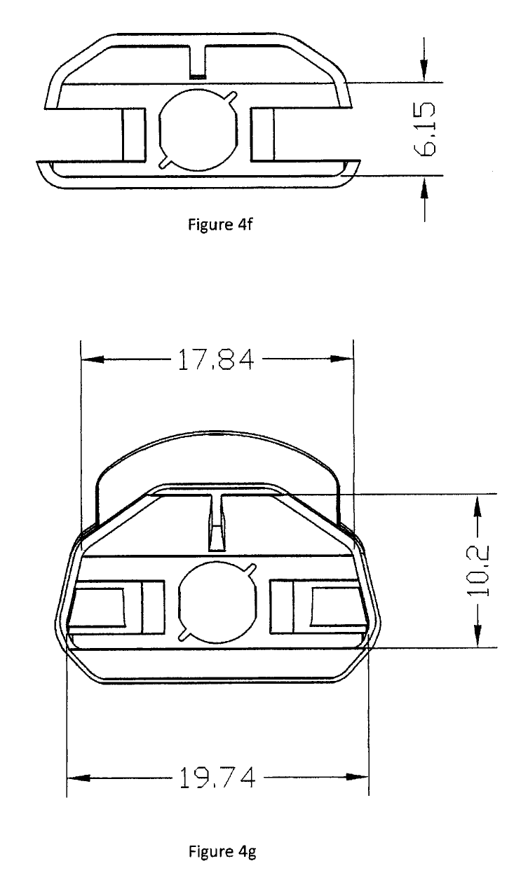

FIG. 4a, FIG. 4b, and FIG. 4c illustrate a side view schematic, a top view schematic, and a front view schematic of the needle shield 100, respectively, according to an embodiment. As illustrated in the examples of FIG. 4a, FIG. 4b, and FIG. 4c, needle shield 100 has tentacles 118, 120 with a side profile length of about 7.5 mm. The length from the front end 116 of needle shield 100 to the tip 402 of a tentacle is approximately 37 mm. Preferably, the angle created by each tentacle with a side face of the needle shield 100 is approximately 27°. The total length of needle shield 100 from base end 112 to front end 116 is approximately 58.0 mm. The two tentacles 118, 120 illustrated in FIG. 4b form traps 404, 406. A ridge 408 running down the middle of the interior side of top face 110 may serve as a guide to retraction. The height of a slot 410 is approximately 3.5 mm.

When the user retracts winged needle 104 to prevent a potential mispiercing after usage, the user pulls back the needle from the front end 116 towards the base end 112. As the winged needle 104 is pulled back from the front end 116 of needle shield 100 to the base end 112 of needle shield 100, the winged needle 104 is in a retracted position when the winged needle 104 has been pulled behind the tentacles 118, 120. Once behind the tentacles 118, 120, the wings of the winged needle 104 can no longer be pushed towards the front end 116. When the user attempts to push the winged needle towards the front end 116, the wings will be caught in the traps formed by the tentacles 118, 120, thus preventing re-protrusion of the needle tube 130.

FIG. 4d illustrates a schematic top view of the needle shield 100, according to one embodiment. As depicted in FIG. 4d, the needle shield 100 has front end 116, base end 112, and tentacles 118, 120. The angle created by each tentacle 118, 120 with a side face of the needle shield 100 is preferably approximately 27°, as indicated in the illustration.

The tentacles illustrated herein represent one embodiment. Other embodiments may have tentacles with different physical features or dimensions. In some embodiments of the invention, traps have a different shape than the shapes depicted in the figures. For example, each of the tentacles may form an angle other than 27° angle with the side face of the needle shield. The tentacles may be curved instead of flat. The tentacles may be textured or smoothed to allow for varying levels of friction with the wings. In some embodiments, each of the tentacles may comprise multiple pieces that are interconnected to create the tentacle. The tentacles may project up from the bottom face of the needle shield, instead of or in addition to projecting from the sides. The tentacles may also project down from the top face of the needle shield. The tentacles may project from any combination of the top face, the bottom face, or the side faces of the needle shield. Embodiments of the invention are not limited to the tentacles depicted in the figures.

FIG. 4e, FIG. 4f, FIG. 4g illustrate an overhead view and schematic diagrams to show dimensions of the needle shield 100, according to an embodiment. FIG. 4e illustrates, for example, the width of bottom face 202 of needle shield 100 is approximately 19.7 mm. The height of needle shield 100 without the arcuate projection 210 is approximately 10.2 mm. The width of top face 110 is approximately 17.8 mm. The height of needle shield 100 at base end 112 is approximately 6.15 mm.

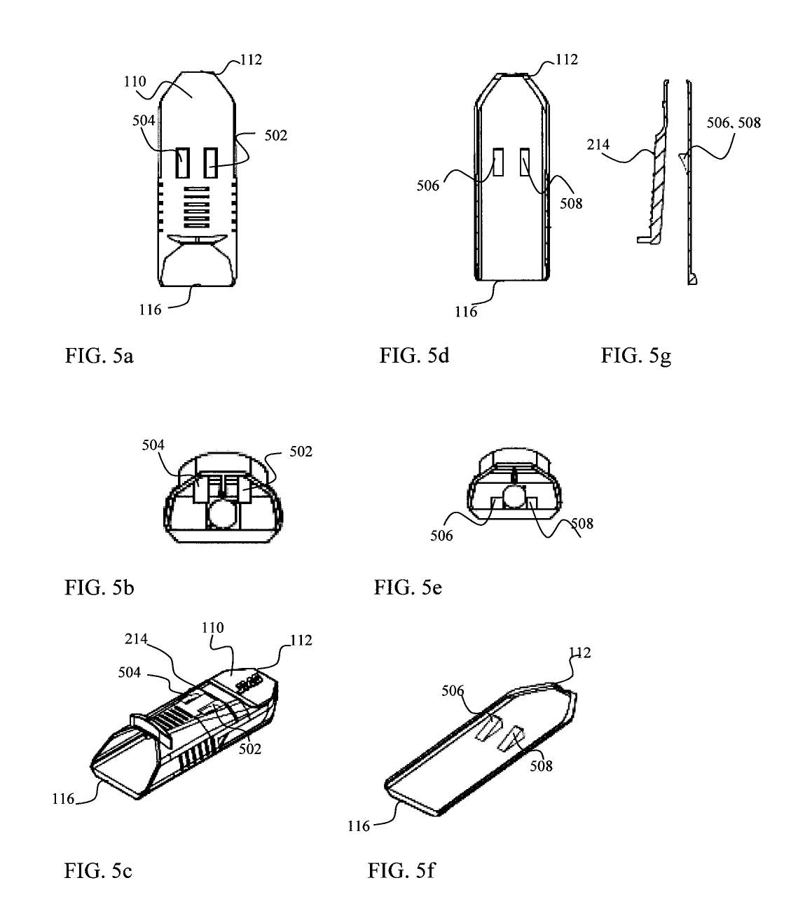

FIGS. 5a, 5b, and 5c illustrate tentacles 502, 504 located at top face 110 of needle shield 100 (FIG. 1) and projecting from top face 110 down into the interior of needle shield 100, according to another embodiment. Tentacles 502, 504 form traps that wings of winged needle 104 slide into when translating towards front end 116, similar to tentacles 118, 120 as described above. Winged needle 104 (FIG. 1) is retracted securely so as to not cause inadvertent mispiercing when the wings 122, 124 have been translated from front end 116 back towards base end 112 to the point that wings 122, 124 are behind the tentacles 502, 504. A safety line 214 located on top face 110 indicates to the user as to how far back towards base end 112 the user translates winged needle 104.

FIGS. 5d, 5e, 5f, and 5g illustrate tentacles 506, 508 located at bottom face 202 of needle shield 100 (FIG. 1) and projecting from bottom face 202 up into the interior of needle shield 100, according to another embodiment. Tentacles 506, 508 form traps that wings of winged needle 104 slide into when translating towards front end 116, similar to tentacles 118, 120 as described above. Winged needle 104 is retracted securely so as to not cause inadvertent mispiercing when the wings 122, 124 have been translated from front end 116 back towards base end 112 to the point that wings 122, 124 are behind the tentacles 506, 508. A safety line 214 indicates to the user as to how far back the user translates winged needle 104.

FIG. 6 illustrates the needle shield 100 with anti-skid notches 602, 604, according to an embodiment. The anti-skid notches 602, 604 allow for a firmer grip when the user is pulling on flexible tube 102 to translate winged needle 104 to a retracted position. Although FIG. 6 depicts needle shield 100 with tentacles 502, 504 projecting from top face 110 down into the interior of needle shield 100, anti-skid notches 602, 604 can be used together with any combination of tentacles projecting from one or more sides of needle shield 100.

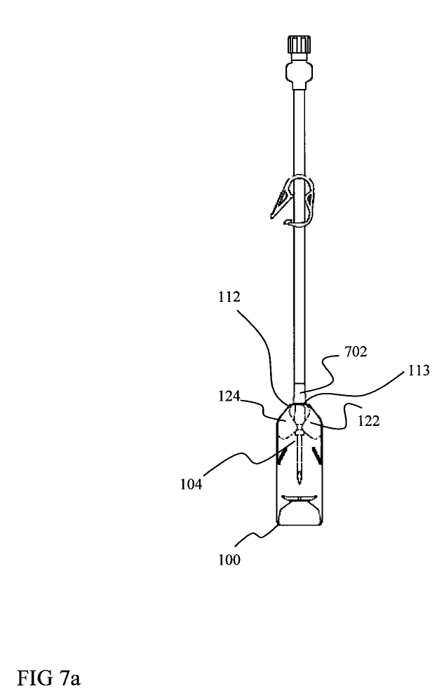

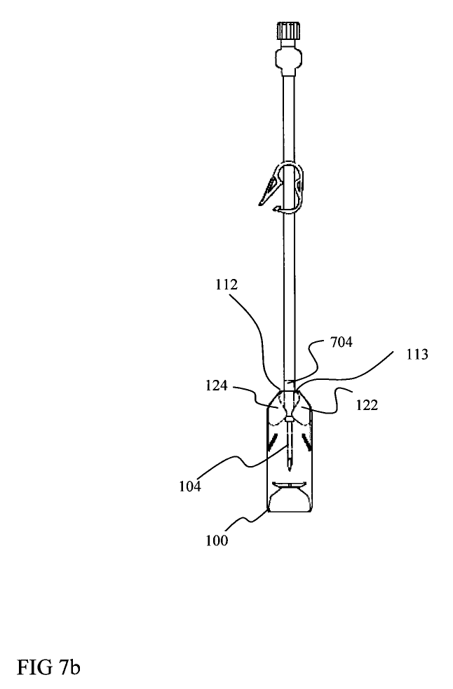

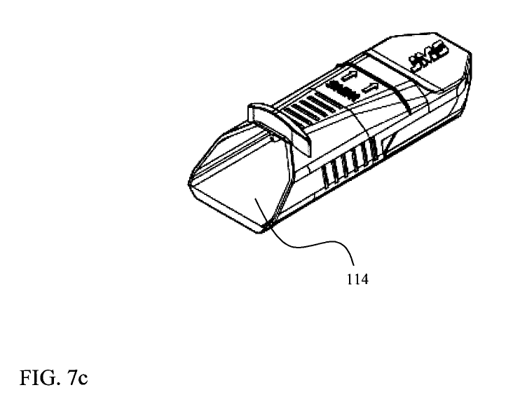

FIG. 7a illustrates needle shield 100 securing a winged needle formed with a rotating hub 702, according to an embodiment. Winged needle 104 can be equipped with the rotating hub 702. Rotating hub 702 allows for wings 122, 124 to rotate. Winged needle 104 is translated towards base end 112 until needle hub 702 extends through circular opening 113. The diameter of circular opening 113 at base end 112 is slightly smaller than the diameter of needle hub 702, causing base end 112 to squeeze and grip a portion of needle hub 702 when needle hub 702 is extended through circular opening 113. Rotating hub 702 is thus grippingly secured by base end 112 when the rotating hub 702 extends through base end 112.

FIG. 7b illustrates needle shield 100 securing a winged needle formed with a wing base 704, according to an embodiment. Winged needle 104 can be equipped with the wing base 704, which does not allow for wings 122, 124 to rotate. Winged needle 104 is translated towards base end 112 until wing base 704 extends through circular opening 113. The diameter of circular opening 113 at base end 112 is slightly smaller than the diameter of wing base 704, causing base end 112 to squeeze and grip a portion of wing base 704 when wing base 704 is extended through circular opening 113. Wing base 704 is thus grippingly secured by base end 112 when wing base 704 extends through circular opening 113.

FIG. 7c illustrates needle shield 100 with additional enhanced features, according to another embodiment. As shown in FIG. 7c, front end opening 114 employs a tapered design to allow for smoother entry of winged needle 104 during retraction. This design can also allow for more efficient production, such as better ejection, during the moulding process.

In one embodiment, needle shield 100 is manufactured through split-mould members integrated by at least one hinge member. In other embodiments, needle shield 100 is advantageously manufactured in a single solid piece form as a whole by moulding. Instead of using a pair of split-mould members integrated by hinge connections, needle shield 100 is produced in one piece, in the example embodiment, and does not have any hinge connections that would allow needle shield 100 to snap apart, whether inadvertently or deliberately. Thus, needle shield 100 cannot be tampered with easily and the winged needle 104 is even more secure. The one-piece moulding design reduces the risk of inadvertent mispiercing due to the reduced risk of exposing needle tube 130 of the winged needle 104. Additionally, needle shield 100 may be integrally formed with winged needle 104 and flexible tube 102.

In example embodiments, with the tentacles trapping the wings of the winged needle 104, advantageously no additional stopper or other obstacle is needed at front end 116 to prevent protrusion of needle tube 130. This can result in manufacturing savings and further reduces costs.

In some embodiments, the needle shield 100 may be made of thermoplastics such as polypropylene, and the internal parts of needle shield 100 in contact with the wing is finished by an embossed process. The wings may have a specific structure or configuration so that the winged needle can be easily retained into the needle shield. The wings may have a structure such that the wings have a tendency to fold upwards. The wings may have a hardness of about 10 to 100 as specified by JIS-A. The wing of the winged needle may be made of vinyl chloride having a hardness of about 70 as specified by JIS-A. The thickness of the wings may be about 1.0 mm, with the material of the wings being soft and easily formed.

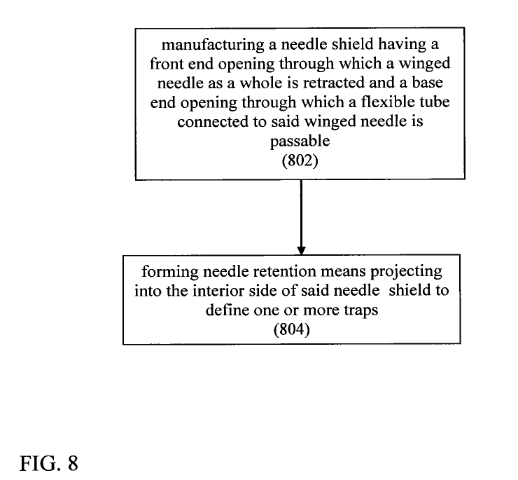

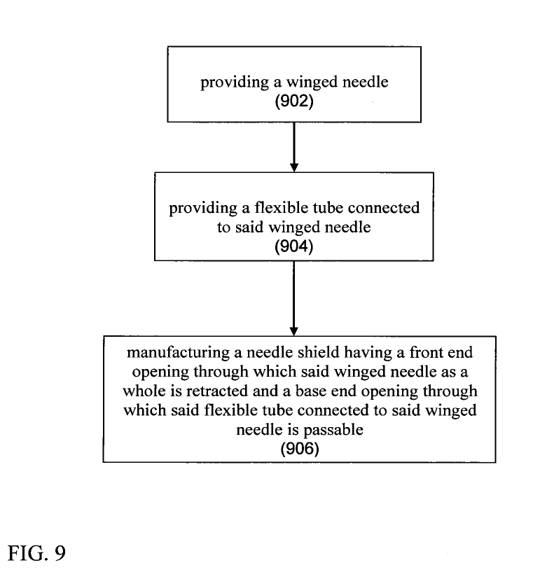

FIG. 8 illustrates a flow diagram for manufacturing a needle shield having a front end opening through which a winged needle as a whole is retracted and a base end opening through which a flexible tube connected to said winged needle is passable (802), according to an example embodiment. At step 804, needle retention means projecting into the interior side of said needle shield to define one or more traps are formed, said winged needle retention means receiving, into said one or more traps, portions of one or more wings of said winged needle for preventing protrusion of said winged needle past said winged needle retention means when said winged needle is translated within said needle shield towards said front end opening of said needle shield from a retracted state of said winged needle. FIG. 9 illustrates a flow diagram for manufacturing a winged needle assembly, according to an example embodiment. At step 902, a winged needle is provided. At step 904, a flexible tube connected to said winged needle is provided. At step 906, a needle shield having a front end opening through which said winged needle as a whole is retracted and a base end opening through which said flexible tube connected to said winged needle is passable is manufactured,

wherein manufacturing the needle shield comprises forming needle retention means projecting into the interior side of said needle shield to define one or more traps, said winged needle retention means receiving, into said one or more traps, portions of one or more wings of said winged needle for preventing protrusion of said winged needle past said winged needle retention means when said winged needle is translated within said needle shield towards said front end opening of said needle shield from a retracted state of said winged needle.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive.

For example, it will be appreciated that embodiments are not limited to the example materials and dimensions described herein. Also, the retention means, e.g. in the form of tentacles, can project from at least one of a side face, a top face, or a bottom face of the needle shield into the interior of said needle shield to define one or more traps in various embodiments.

The invention claimed is:

1. A needle shield having a front end opening through which a winged needle as a whole is retracted and a base end opening through which a flexible tube connected to said winged needle is passable, said needle shield comprising:
winged needle retention means projecting into the interior side of said needle shield to define one or more traps, and
wherein said winged needle retention means receiving, into said one or more traps, portions of one or more wings of said winged needle for preventing protrusion of said winged needle past said winged needle retention means when said winged needle is translated within said needle shield towards said front end opening of said needle shield from a retracted state of said winged needle.

2. The needle shield according to claim 1, wherein said winged needle retention means projects from at least one of a side face, a top face, or a bottom face of said needle shield into the interior of said needle shield to define one or more traps.

3. The needle shield according to claim 2, wherein said one or more traps is each formed by a tentacle joined to one of said side face, said top face, or said bottom face of said needle shield.

4. The needle shield according to claim 3, wherein at least one of said one or more traps is formed by creating an acute angle.

5. The needle shield according to claim 1, further comprising a propping means propping said needle shield away from skin of a user of said needle shield.

6. The needle shield according to claim 5, wherein said propping means is a protrusion with radius edges protruding from an exterior side of a bottom face of said needle shield.

7. The needle shield according to claim 1, wherein said base end opening grippingly secures a needle hub or a wing base of said winged needle when said winged needle is translated within said needle shield towards said base end opening.

8. A winged needle assembly comprising:
a winged needle;
a flexible tube connected to said winged needle, and
a needle shield integrally formed with said flexible tube and having a front end opening through which said winged needle as a whole is retracted and a base end opening through which said flexible tube connected to said winged needle is passable, wherein one or more tentacles project into the interior side of said needle shield to define one or more traps, wherein said one or more tentacles are capable of receiving, into said one or more traps, portions of one or more wings of said winged needle, wherein said needle shield prevents protrusion of said winged needle past said one or more tentacles when said winged needle is translated within said needle shield towards said front end opening of said needle shield from a retracted state of said winged needle.

9. A method of manufacturing a needle shield having a front end opening through which a winged needle as a whole is retracted and a base end opening through which a flexible tube connected to said winged needle is passable, the method comprising the steps of:
forming needle retention means projecting into the interior side of said needle shield to define one or more traps,
said winged needle retention means receiving, into said one or more traps, portions of one or more wings of said winged needle for preventing protrusion of said winged needle past said winged needle retention means when said winged needle is translated within said needle shield towards said front end opening of said needle shield from a retracted state of said winged needle.

10. The method according to claim 9, wherein said needle shield is manufactured in a single solid piece form as a whole by moulding.

11. The method according to claim 9, wherein said needle shield is manufactured through split-mould members integrated by at least one hinge member.

12. A method of manufacturing a winged needle assembly, the method comprising the steps of:
providing a winged needle;
providing a flexible tube connected to said winged needle; and
manufacturing a needle shield having a front end opening through which said winged needle as a whole is retracted and a base end opening through which said flexible tube connected to said winged needle is passable,
wherein manufacturing the needle shield comprises forming needle retention means projecting into the interior side of said needle shield to define one or more traps, said winged needle retention means receiving, into said one or more traps, portions of one or more wings of said winged needle for preventing protrusion of said winged needle past said winged needle retention means when said winged needle is translated within said needle shield towards said front end opening of said needle shield from a retracted state of said winged needle.

13. The method according to claim 12, wherein said needle shield is manufactured in a single solid piece form as a whole by moulding.

14. The method according to claim 12, wherein said needle shield is manufactured through split-mould members integrated by at least one hinge member.
